(19) United States
(12) Patent Application Publication
(10) Pub. No.: US 2022/0347119 A1
(43) Pub. Date: Nov. 3, 2022

(54) MEDICATED DRINK

(71) Applicant: Reshma N. Kheraj, Raleigh, NC (US)

(72) Inventor: Reshma N. Kheraj, Raleigh, NC (US)

(21) Appl. No.: 17/721,840

(22) Filed: Apr. 15, 2022

Related U.S. Application Data
(60) Provisional application No. 63/182,630, filed on Apr. 30, 2021.

(51) Int. Cl. A61K 31/05; A61K 31/4045; A61K 31/197; A61K 31/198; A61K 36/35; A61K 9/107; A61K 31/352; A61K 33/00

(52) U.S. Cl. CPC A61K 31/05; A61K 31/4045; A61K 31/197; A61K 31/198; A61K 36/35; A61K 9/1075; A61K 31/352; A61K 33/00

(57) ABSTRACT

A medicated drink for relieving stress and helping a person with insomnia to fall asleep faster. The medicated drink includes Cannabidiol (CBD); Melatonin; GABA (Gamma-Aminobutyric-acid); L-Theanine-Amino acid; Valerian Root-aqueous extract; and Water.

### CROSS-REFERENCE TO RELATED APPLICATIONS

This application claims priority from the U.S. provisional patent application Ser. No. 63/182,630, filed on Apr. 30, 2021, which is incorporated herein by reference in its entirety

### FIELD OF INVENTION

The present invention relates to medicated drink, and more particularly, the present invention is related to drinkable water infused with medicated nano-emulsion.

### BACKGROUND

A healthy mental condition is essential for the overall well-being of an individual. However, many mental disorders, such as anxiety and insomnia have become quite common. The primary reason for such mental disorders is the modern lifestyle.

Several drinks are available for consumption by humans that may offer different health benefits, such as energy, nutrition, and the like. The drinks have the advantage that they are readily available for consumption, and sometimes in place of drinking water.

A desire is there for a drink that can soothe the mind and help a person fall asleep.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a medicated drink for consumption by humans that can soothe an anxious mind.

It is another object of the present invention that the medicated drink can help an insomniac person to sleep.

It is still another object of the present invention that the medicated drink has nutritious value as well.

It is yet another object of the present invention that the medicated drink is made from natural ingredients.

It is a further object of the present invention that the medicated drink is economical to manufacture.

It is still another object of the present invention that the bioabsorption of active ingredients in the medicated drink is enhanced.

It is an additional object of the present invention that the medicated drink can have a good shelf life.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, the reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an”, and “the” are intended to include the plural forms as well, unless the context indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed is a medicated drink for consumption by humans. The medicated drink can help a person relax, in particular, the medicated drink can have anti-anxiety effects. The disclosed medicated drink can also be useful for insomniac people, i.e, people which find it difficult to sleep. The disclosed medicated drink can help such persons with insomnia to sleep. Besides helping a person to fall asleep faster, the disclosed medicated drink can also help the person to get a good sleep.

This is drinkable water that is infused with ingredients that help a person relax, fall asleep faster, and maintain a good night's sleep.

The disclosed medicated drink can include CBD which can help improve the circadian rhythms of an individual and can also help the person fall asleep faster and maintain a good overnight sleep. The CBD can be incorporated in nano form that can better cross the blood-brain barrier for a more potent effect. The amount of nano CBD per drink can be about 250-1000 mg. The disclosed medicated drink can further include melatonin which is a natural hormone and promotes sleep. The melatonin can optionally be added in nano form to improve bioavailability at the site of action. About 5-10 mg of the nano melatonin can be incorporated into a medicated drink. The disclosed medicated drink can also include GABA (Gamma-Aminobutyric-acid) which is a naturally occurring amino acid that works as an inhibitory neurotransmitter in the brain. It helps relieve anxiety and improve sleep. About 200-750 mg of GABA can be incorporated into a single medicated drink. The disclosed medicated drink can also include L-Theanine-Amino acid which helps ease anxiety, and stress and reduce insomnia. About 50-200 mg of L-Theanine-Amino can be incorporated. The disclosed medicated drink can further include Valerian Root-aqueous extract, which is a Herb that can be effective against insomnia and nervousness. About 200-1000 mg of Valerian Root-aqueous extract can be incorporated into the disclosed medicated drink.

The disclosed medicated drink includes water into which the above ingredients can be added. The water can additionally contain minerals, flavors, edible coloring substances, and the like without departing from the scope of the present invention. In certain implementation, the disclosed medicated drink can also have tea infused (Sleep Tea) to it, for example, Chamomile Tea. Water is the medium for other ingredients and can also fulfill the hydration requirements of the body. The active ingredients in the water can be about 705-2960 mg per about 500 ml of the medicated drink.

Besides the above ingredients, additional ingredients can also be added, such as 5-hydroxytryptophan (L-5-HTP) amino acid, Magnesium, Potassium, Sodium, Glucose, Dextrose, Vitamin-B6, Turmeric, and Zinc. The additional ingredients may have certain health benefits and can be added in suitable proportions without departing from the scope of the present invention. For example, Sodium, Magnesium, Potassium, Glucose, and water will help quicken the absorption of the main active ingredients into the bloodstream to get faster efficacy.

The water-soluble active ingredients can be directly added to water and get dissolved. Hydrophobic active ingredients, such as CBD, can be processed to be added to the water, further, to have a good shelf life. For example, CBD can be added as nanodroplets, wherein the CBD extracts can be subjected to ultrasonic processors/sheering, wherein the use of sheer force of the ultrasonic cavitation can reduce the droplet size to nano size. The ultrasonics can create water-dispersible translucent nano-emulsions that help increase bioavailability and ensure more accurate dosing. The water infused with nano-emulsions can offer enhanced absorption and bioavailability of the active ingredients and will offer rapid onset of the effects of the main active ingredients. Also, the drink appears homogenous and has a certain degree of optical clarity.

In certain implementations, the nano-emulsions can be made from the mixture of purified water, CBD, Melatonin, and oil-in-water surfactant for emulsification using a suitable process.

The disclosed medicated drink can be packed in standard packaging, such as bottles, cans, and tetra packs. The packings of different volumes can be provided including 250 ml, 300 ml, 500 ml, and the like. The bottles can be made of plastic or glass. Preferably opaque packaging can be used to prevent light-mediated degradation of the active ingredients.

In certain implementations, the disclosed medicated drink can also be infused with nitrogen gas. In certain implementations, the medicated drink can also include cannabis, preferably, about 10-100 mg of THC.

What is claimed is:

1. A medicated drink comprising:
cannabidiol (CBD);
melatonin;
GABA (Gamma-Aminobutyric-acid);
L-Theanine amino acid;
valerian root-aqueous extract; and
water.

2. The medicated drink according to claim 1, wherein:
cannabidiol (CBD): 250-1000 mg;
Melatonin: 5-10 mg;
GABA: 200-750 mg;
L-Theanine amino acid: 50-200 mg;
Valerian Root-aqueous extract: 200-1000 mg; and
Water: quantity sufficient (QS).

3. The medicated drink according to claim 1, wherein the CBD is present in a nano-emulsion form.

4. The medicated drink according to claim 1, wherein the medicated drink further comprises nitrogen gas.

5. The medicated drink according to claim 1, wherein the medicated drink further comprises Tetrahydrocannabinol (THC).

6. The medicated drink according to claim 2, wherein 500 ml of water is present.

7. A method of soothing an anxious mind or helping a person to fall asleep, the method comprises the steps of:
providing a medicated drink comprising:
cannabidiol (CBD),
melatonin,
GABA (Gamma-Aminobutyric-acid),
L-Theanine amino acid,
valerian root-aqueous extract; and
water.

8. The method according to claim 7, wherein the CBD is present in a nano-emulsion form.

9. The method according to claim 7, wherein the medicated drink further comprises nitrogen gas.

10. The method according to claim 7, wherein the medicated drink further comprises Tetrahydrocannabinol (THC).

11. The method according to claim 7, wherein 500 ml of water is present.
